(19) United States
(12) Patent Application Publication
(10) Pub. No.: US 2009/0036550 A1
(43) Pub. Date: Feb. 5, 2009

(54) Copolymers Based on Polyalkylene Oxide-Modified N-Vinyl Lactam Copolymers

(75) Inventors: Nathalie Bouillo, Baden-Baden (DE); Alexander Khvorost, Laudenbach (DE); Ingo Munster, Bohl-Iggelheim (DE); Rainer Dobrawa, Mannheim (DE); Kathrin Meyer-Bohm, Feucht (DE); Ronald Frans Maria Lange, Ludwigshafen (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/096,527

(22) PCT Filed: Nov. 30, 2006
(86) PCT No.: PCT/EP2006/069128

§ 371 Date: Jun. 6, 2008

(30) Foreign Application Priority Data
Dec. 9, 2005 (EP) 05111921.2

(51) Int. Cl. A61K 47/32; C08F 26/06

(52) U.S. Cl. 514/772.5; 526/264

(57) ABSTRACT

Vinyllactam copolymers modified with polyalkylene oxide side-chains, and use thereof as solubilizers for substances which are sparingly soluble in water, and preparations comprising such copolymers.

The invention relates to N-vinyllactam copolymers modified with polyalkylene oxide side-chains, to the production thereof and to the use thereof as solubilizers of biologically active substances which are sparingly soluble in water. In addition, the invention relates to corresponding preparations for use on humans, animals and plants.

When producing homogeneous preparations of biologically active substances, the solubilization of hydrophobic substances, i.e. substances which are sparingly soluble in water, has achieved very great practical importance.

Solubilization is understood as meaning making substances which are insoluble or sparingly soluble in a certain solvent, in particular water, soluble through interface-active compounds, the solubilizers. Such solubilizers are able to convert poorly water-soluble or water-insoluble substances into clear, at most opalescent aqueous solutions without the chemical structure of these substances undergoing a change in the process (cf. Römpp Chemie Lexikon, 9^{th} edition, Vol. 5, p. 4203, Thieme Verlag, Stuttgart, 1992).

The prepared solubilizates are notable for the fact that the poorly water-soluble or water-insoluble substance is present in colloidally dissolved form in the molecular associates of the surface-active compounds which form in aqueous solution—the so-called micelles. The resulting solutions are stable single-phase systems which appear to be visually clear to opalescent and can be prepared without the input of energy.

Solubilizers can, for example, improve the appearance of cosmetic formulations and of food preparations by making the formulations transparent. Furthermore, in the case of pharmaceutical, preparations, the bioavailability and thus the effect of medicaments can also be increased through the use of solubilizers.

The solubilizers used for pharmaceutical medicaments and cosmetic active ingredients are primarily surfactants such as ethoxylated (hydrogenated) castor oil, ethoxylated sorbitan fatty acid esters or ethoxylated hydroxystearic acid.

However, the hitherto used solubilizers described above have a number of application-related disadvantages.

The known solubilizers have only a small solubilizing effect for some sparingly soluble medicaments such as, for example, clotrimazole.

In addition, the solubilizers known hitherto are mostly liquid or semisolid compounds which, on account of this, have relatively unfavorable processing properties.

U.S. Pat. No. 4,432,881 describes hydrophobically modified polyacrylic acid with a molecular weight between 200000 and 5000000 which are obtained by copolymerization of acrylic acid with the corresponding N-alkylacrylamides or acrylates. The polymers obtained are used as dispersible hydrophobic thickeners.

U.S. Pat. No. 4,395,524 describes the copolymerization of hydrophilic components (e.g. acrylamide, acrylic acid, N-vinylpyrrolidone etc.) with N-alkylacrylamides. The polymers obtained in this way with a molecular weight of from 30000 to 2000000 are used as thickeners, sedimentation stabilizers or dispersants.

EP-A-0 268 164 describes the use of copolymers of monoolefinically unsaturated acids and alkyl esters of monoolefinically unsaturated acids for stabilizing O/W emulsions.

EP-A 876 819 describes the use of copolymers of at least 60% by weight of N-vinylpyrrolidone and amides or esters with long-chain alkyl groups as solubility promoters for sparingly soluble active ingredients.

EP-A 948 957 describes the use of copolymers of monoethylenically unsaturated carboxylic acids such as, for example, acrylic acid and hydrophobically modified comonomers such as, for example, N-alkyl- or N,N-dialkylamides of unsaturated carboxylic acids with C₈-C₃₀-alkyl radicals as solubility promoters for sparingly soluble active ingredients.

The object was to provide novel solubilizers for pharmaceutical, cosmetic, food and agrotechnical applications.

This object was achieved by the copolymers defined at the start which are obtained by reacting a hydroxyl or amino-functionalized N-vinyllactam copolymer with polyalkylene oxides or by direct polyalkoxylation of the hydroxy or amino groups of the N-vinyllactam copolymer.

In addition, the invention provides the use thereof as solubilizers for substances which are sparingly soluble in water, and corresponding preparations.

The vinyllactam copolymers used as prepolymers are obtained by free-radically initiated copolymerization of from 50 to 99.9 mol % of N-vinyllactams with 0.1 to 50 mol % of a further monoolefinically unsaturated comonomer. Preference is given to using 80 to 99 mol % of N-vinyllactam and 1 to 20 mol % of the comonomer.

Suitable N-vinyllactams are N-vinylpyrrolidone, N-vinylpiperidone and N-vinylcaprolactam or mixtures of these monomers. A preferred N-vinyllactam is N-vinylpyrrolidone.

Suitable comonomers are monoolefinically unsaturated monomers which carry hydroxyl or amino groups or comprise a group which can be converted into hydroxyl or amino groups by hydrolysis.

Accordingly, suitable comonomers are:

vinyl esters of C₂-C₈-carboxylic acids, such as vinyl acetate or vinyl propionate, monovinyl ethers of C₂-C₈-diols, for example ethylene glycol monovinyl ether, diethylene glycol monovinyl ether, 1,4-butanediol monovinyl ether, 1,6-hexanediol monovinyl ether or 1,4-clohexanedimethanol monovinyl ether, N-vinylamides, such as, for example, N-vinylacetamide or N-vinylformamide, hydroxyalkyl esters or aminoalkyl esters of acrylic acid or of methacrylic acid with C₁-C₈-alkyl chain lengths, such as, for example, hydroxyethyl methacrylate.

Preferred comonomers are vinyl-acetate, vinyl propionate and N-vinylformamide, very particularly preferably vinyl acetate.

The prepolymers are produced by methods known per se, e.g. by solution polymerization, precipitation polymerization or by inverse emulsion polymerization, in the presence of compounds which form free radicals under the polymerization conditions.

The polymerization temperatures are usually in the range from 30 to 200° C., preferably 40 to 110° C. Suitable initiators are, for example, azo and peroxy compounds, and the customary redox initiator systems, such as combinations of hydrogen peroxide and compounds with a reducing effect, e.g. sodium sulfite, sodium bisulfite, sodium formaldehyde sulfoxylate and hydrazine.

The reaction medium used is any customary solvent in which the monomers are soluble. Preferably, water or alcoholic solvents, such as, for example, methanol, ethanol, n-propanol or isopropanol or mixtures of such alcohols with water, are used.

In order to ensure that the reaction leads to homogeneous products, it is advantageous to supply the monomers and the initiator separately to the reaction solution. This can take place, for example, in the form of separate feeds for the individual reactants.

The polymerization can also be carried out in the presence of customary regulators if relatively lower molecular weights are to be established.

A nonaqueous solvent used for the polymerization can then be removed by means of steam distillation and be replaced by water.

After the copolymerization, a saponification takes place, if appropriate, with formation of the corresponding hydroxy or amino functions. The saponification can be carried out in a manner known per se by reaction with bases, such as, for example, alcoholic solutions of hydroxides, such as sodium hydroxide or potassium hydroxide, or alcoholic solutions of alkoxides, such as, for example, methanolic sodium or potassium methoxide solution or ethanolic sodium or potassium ethoxide solution.

The prepolymers obtained in this way usually have molecular weights Mw of from 10,000 to 150,000 g/mol.

According to a preferred embodiment of the invention, the prepolymers obtained in this way are reacted with polyalkylene oxides.

Suitable polyalkylene oxides are preferably polyalkylene glycols. The polyalkylene glycols can have molecular weights of from 500 to 10,000 D [daltons], preferably 1,000 to 7,500 D, particularly preferably 1,000 to 5,000 D. The molecular weights are determined starting from the hydroxyl number measured in accordance with DIN 53240.

Suitable particularly preferred polyalkylene glycols are polyethylene glycols. Furthermore, polypropylene glycols, polytetrahydrofurans or polybutylene glycols which are obtained from 2-ethyloxirane or 2,3-dimethyloxirane are also suitable.

Suitable polyethers are also random or blocklike copolymers of polyalkylene glycols obtained from ethylene oxide, propylene oxide and butylene oxide, such as, for example, polyethylene glycol-polypropylene glycol block copolymers. The block copolymers can be of the AB or ABA type.

The preferred polyalkylene glycols also include those which are substituted on one of the two OH end groups. Suitable substituents are alkyl, aryl or aralkyl radicals having 1 to 30 carbon atoms. Suitable aryl radicals are phenyl, naphthyl radicals. Suitable aralkyl radicals are, for example, benzyl radicals. Suitable alkyl radicals are branched or unbranched, open-chain or cyclic C₁- to C₂₂-alkyl radicals. Suitable cycloalkyl radicals are, for example, cyclopentyl, cyclohexyl, cycloheptyl or cyclooctyl radicals which, if appropriate, may be substituted by one or more C₁-C₄-alkyl radicals. Preferably, C₁-C₁₈-alkyl radicals, for example methyl, ethyl, n-butyl, isobutyl, pentyl, hexyl, octyl, nonyl, decyl, dodecyl, tridecyl or octadecyl radicals, are suitable.

Such polyalkylene oxides are either easy to prepare or commercially available.

The polyalkylene oxides are reacted in equimolar amounts, based on the amino or hydroxyl groups in the prepolymer. The amount of OH and amino groups present can, if required, take place in a manner known per se to the person skilled in the art. To determine the hydroxyl number, see, for example, Römpp Chemie Lexikon, 9th edition, 1990.

According to one embodiment of the invention, the reaction of the vinyllactam copolymers with polyalkylene oxides in the case of hydroxyl-functionalized vinyllactam copolymers can take place by direct alkoxylation of the OH functions of the copolymer. The direct alkoxylation can take place by reacting the copolymer with the corresponding monomeric alkylene oxide in the presence of a catalyst. Preference is given to using potassium hydroxide as catalyst. Suitable catalysts are also alkali metal hydroxides, such as sodium or potassium hydroxide, montmorillonite, acidic ion exchangers, Lewis acids, such as boron trifluoride or double-metal cyanides, such as zinc hexacyanoferrate. The reaction can take place in an organic solvent, preferably a polar aprotic solvent, very particularly preferably in dimethylformamide. The reaction can take place at temperatures of from 100 to 180° C. The reaction is preferably carried out in a pressurized reactor.

A further preferred embodiment of the invention provides the coupling of vinyllactam copolymers and polyalkylene oxides by reaction with diisocyanates, where reaction with the hydroxyl or amino groups of the vinyllactam copolymer leads to the polyalkylene oxide coupling onto the vinyllactam copolymer via urethane or urea groups. Here, either the vinyllactam copolymer or the polyalkylene oxide can firstly be reacted with the diisocyanate.

According to a preferred embodiment of the invention, the coupling takes place via polyalkylene oxides functionalized with isocyanate groups as end groups. For this, firstly the polyalkylene oxide is reacted with the diisocyanate and then the polyalkylene oxide functionalized in this way is reacted with the copolymer.

Irrespective of which embodiment is chosen, the reaction can be carried out as follows:

Suitable diisocyanates are compounds of the general formula OCN—R—NCO, where R can be aliphatic, alicyclic or aromatic radicals which may also be substituted by alkyl radicals.

Suitable diisocyanates are preferably compounds whose isocyanate groups have, on account of the molecular structure, a different reactivity toward nucleophiles, for example isophorone diisocyanate or tolylene diisocyanate.

Also suitable in principle are symmetrical diisocyanates, such as, for example, hexamethylene diisocyanate or 4,4′-methylenedi(phenyl isocyanate).

Preference is given to using isophorone diisocyanate.

The reaction with the diisocyanate takes place preferably in an organic solvent, such as ketones, for example acetone, also dimethyl sulfoxide, dimethylformamide, or generally aprotic-polar organic solvents or mixtures of such solvents. The reaction takes place usually at elevated temperatures, the temperature also being governed by the boiling temperature of the solvent chosen. The reaction of the diisocyanate with the first component can take place at 20 to 50° C., but if desired also to 100° C. The reaction of the second isocyanate group can take place at temperatures of from 50 to 100° C.

The reaction preferably takes place equimolarly, which means that the quantitative ratio is chosen so that preferably 1 mol of diisocyanate is used per mole of hydroxyl or amino function to be reacted.

In the case of symmetrical diisocyanates, it may also be advisable to use an excess of diisocyanate and then to remove the excess by distillation.

Preferably, the reaction is carried out in the presence of a catalyst. Suitable catalysts are, for example, organometallic compounds, such as organotitanium compounds or zinc compounds, such as dibutyltin dilaurate or tin octoate, also bases, such as 1,4-diaza(2.2.2)bicyclooctane or tetramethylbutanediamine. The catalyst can be used in amounts of from 0.05 to 0.2 mol, preferably 0.1 to 0.1 mol, per mole of diisocyanate.

The reaction is usually carried out at elevated temperatures in the range from 50 to 100° C. Which temperature is chosen in a specific case depends on the type of organic solvent used. The solvent can then be removed by distillation.

Usually, the reaction is carried out in a way which involves firstly reacting the component, which should be isocyanate-group-functonalized, with the diisocyanate in the presence of the catalyst and a solvent until the isocyanate value in the reaction mixture has dropped to half. This can be ascertained by known methods, for example titrimetrically. Afterwards, the other component is then added, with the amounts of isocyanate groups and OH or amino groups again being chosen to be equimolar. The reaction is continued until the isocyanate value has dropped to zero.

If polyalkylene oxides are not to be coupled to all of the hydroxyl or amino functions of the vinyllactam copolymer, it is also possible to use a deficit of isocyanate-group-functionalized polyalkylene oxides.

The resulting copolymers based on vinyllactam copolymers and polyalkylene oxides are water-soluble or water-dispersible. Preference is given to water-soluble copolymers.

The molecular weights M_{w} can be 20,000 to 500,000 g/mol, preferably 20,000 to 250,000, particularly preferably 80,000 to 250,000 g/mol. The molecular weights can be determined with the help of gel chromatography.

### APPLICATIONS

The copolymers to be used according to the invention can in principle be used in all fields where substances which are insoluble or only sparingly soluble in water are either to be used in aqueous preparations or are to develop their effect in an aqueous medium. Accordingly, the copolymers are used as solubilizers of substances which are sparingly soluble in water, in particular biologically active substances.

According to the invention, the term “sparingly soluble in water” also comprises virtually insoluble substances and means that, for a solution of the substance in water at 20° C., at least 30 to 100 g of water per g of substance are required. In the case of virtually insoluble substances, at least 10 000 g of water per 9 of substance are required.

For the purposes of the present invention, biologically active substances which are sparingly soluble in water are understood as meaning pharmaceutical active ingredients for humans and animals, cosmetic or agrochemical active ingredients or food supplements or dietetic active ingredients.

In addition, suitable sparingly soluble substances to be solubilized are also dyes, such as inorganic or organic pigments.

By virtue of the present invention, amphiphilic compounds in particular for use as solubilizers for pharmaceutical and cosmetic preparations and also for food preparations are provided. They have the property of solubilizing sparingly soluble active ingredients in the fields of pharmacy and cosmetics, sparingly soluble food supplements, for example vitamins and carotenoids, but also sparingly soluble active ingredients for use in crop protection compositions, and also veterinary medicine active ingredients.

### Solubilizers for Cosmetics:

According to the invention, the copolymers can be used as solubilizers in cosmetic formulations. For example, they are suitable as solubilizers for cosmetic oils. They have a good solubilizing ability for fats and oils, such as peanut oil, jojoba oil, coconut oil, almond oil, olive oil, palm oil, castor oil, soybean oil or wheatgerm oil or for essential oils, such as dwarf pine oil, lavender oil, rosemary oil, spruce needle oil, pine needle oil, eucalyptus oil, peppermint oil, sage oil, bergamot oil, turpentine oil, melissa oil, sage oil, juniper oil, lemon oil, anise oil, cardamom oil; peppermint oil, camphor oil etc. or for mixtures of these oils.

In addition, the polymers according to the invention can be used as solubilizers for UV absorbers which are insoluble or sparingly soluble in water, such as, for example, 2-hydroxy-4-methoxybenzophenone (Uvinul® M 40, BASF), 2,2′,4,4′-tetrahydroxybenzophenone (Uvinul® D 50), 2,2′-dihydroxy-4,4′-dimethoxybenzophenone (Uvinul® D49), 2,4-dihydroxybenzophenone (Uvinul® 400), 2′-ethylhexyl 2-cyano-3,3-diphenylacrylate (Uvinul® N 539), 2,4,6-trianilino-p-(carbo-2′-ethylhexyl-1′-oxy)-1,3,5-triazine (Uvinul® T 150), 3-(4-methoxybenzylidene)camphor (Eusolex® 6300, Merck), 2-ethylhexyl N,N-dimethyl-4-aminobenzoate (Eusolex® 6007), 3,3,5-trimethylcyclohexyl salicylate, 4-isopropyldibenzoylmethane (Eusolex® 8020), 2-ethylhexyl p-methoxycinnamate and 2-isoamyl p-methoxycinnamate, and mixtures thereof.

The present invention therefore also provides cosmetic preparations which comprise at least one of the copolymers according to the invention of the composition specified at the start as solubilizers. Preference is given to those preparations which, besides the solubilizer, comprise one or more sparingly soluble cosmetic active ingredients, for example the abovementioned oils or UV absorbers.

These formulations are solubilizates based on water or water/alcohol. The solubilizers according to the invention are used in the ratio from 0.2:1 to 20:1, preferably 1:1 to 15:1, particularly preferably 2:1 to 12:1 relative to the sparingly soluble cosmetic active ingredient.

The content of solubilizer according to the invention in the cosmetic preparation is, depending on the active ingredient, in the range from 1 to 50% by weight, preferably 3 to 40% by weight, particularly preferably 5 to 30% by weight.

In addition, further auxiliaries can be added to this formulation, for example nonionic, cationic or anionic surfactants, such as alkyl polyglycosides, fatty alcohol sulfates, fatty alcohol ether sulfates, alkanesulfonates, fatty alcohol ethoxylates, fatty alcohol phosphates, alkylbetaines, sorbitan esters, POE sorbitan esters, sugar fatty acid esters, fatty acid polyglycerol esters, fatty acid partial glycerides, fatty acid carboxylates, fatty alcohol sulfosuccinates, fatty acid sarcosinates, fatty acid isethionates, fatty acid taurinates, citric acid esters, silicone copolymers, fatty acid polyglycol esters, fatty acid amides, fatty acid alkanolamides, quaternary ammonium compounds, alkylphenol oxethylates, fatty amine oxethylates, cosolvents, such as ethylene glycol, propylene glycol, glycerol etc.

Further constituents which may be added are natural or synthetic compounds, e.g. lanolin derivatives, cholesterol derivatives, isopropyl myristate, isopropyl palmitate, electrolytes, dyes, preservatives, acids (e.g. lactic acid, citric acid).

These formulations are used, for example, in bath additive preparations such as bath oils, aftershaves, face tonics, hair tonics, eau de Cologne, eau de toilette and in sunscreen compositions. A further field of use for polymers of this type is the oral care sector, for example in mouthwashes, toothpastes, adhesive creams for dentures and the like.

In addition, the copolymers are also suitable for industrial applications, for example for preparations of sparingly soluble colorants, in toners, preparations of magnetic pigments and the like.

### Description of the Solubilization Method:

In the preparation of the solubilizates for cosmetic formulations, the copolymers according to the invention can be used as 100% strength substance or preferably as aqueous solution.

Usually, the solubilizer is dissolved in water and vigorously mixed with the sparingly soluble cosmetic active ingredient to be used in each case.

However, it is also possible to vigorously mix the solubilizer with the sparingly soluble cosmetic active ingredient to be used in each case and then to add demineralized water with continuous stirring.

### Solubilizers for Pharmaceutical Applications:

The claimed copolymers are likewise suitable for use as solubilizer in pharmaceutical preparations of any type which are notable for the fact that they can comprise one or more medicaments which are insoluble or sparingly soluble in water, and also vitamins and/or carotenoids. In particular, these are aqueous solutions or solubilizates for oral application.

Thus, the claimed copolymers are suitable for use in oral administration forms such as tablets, capsules, powders, solutions. Here, they can make the sparingly soluble medicament available with increased bioavailability.

In the case of parenteral application, it is also possible to use emulsions, for example fatty emulsions, besides solubilizates. The claimed copolymers are also suitable for this purpose, in order to process a sparingly soluble medicament.

Pharmaceutical formulations of the abovementioned kind can be obtained by processing the claimed copolymers with pharmaceutical active ingredients by conventional methods and with the use of known and novel active ingredients.

The use according to the invention can additionally comprise pharmaceutical auxiliaries and/or diluents. Cosolvents, stabilizers, preservatives are especially mentioned as auxiliaries.

The pharmaceutical active ingredients used are substances which are insoluble or slightly soluble in water. According to DAB 9 (German Pharmacopoeia), the solubility of pharmaceutical active ingredients is categorized as follows: slightly soluble (soluble in 30 to 100 parts of solvent); sparingly soluble (soluble in 100 to 1000 parts of solvent); virtually insoluble (soluble in more than 10 000 parts of solvent). The active ingredients can here come from any area of indication.

Examples which may be mentioned here are benzodiazepines, antihypertensives, vitamins, cytostatics, in particular taxol, anesthetics, neuroleptics, antidepressants, antibiotics, antimycotics, antiviral agents, fungicides, chemotherapeutics, urologics, thrombocyte aggregation inhibitors, sulfonamides, spasmolytics, hormones, immunoglobulins, sera, thyroid therapeutic agents, psychopharmacological agents, antiParkinsonians and other antihyperkinetic agents, ophthalmics, neuropathy preparations, calcium metabolism regulators, muscle relaxants, narcotics, antilipemics, hepatic therapeutic agents, coronary agents, cardiacs, immunotherapeutics, regulatory peptides and their inhibitors, hypnotics, sedatives, gynecological agents, antigouts, fibrinolytic agents, enzyme preparations and transport proteins, enzyme inhibitors, emetics, circulation-promoting agents, diuretics, diagnostics, corticoids, cholinergics, bile duct therapeutics, antiasthmatics, broncholytics, beta-receptor blockers, calcium antagonists, ACE inhibitors, antiarteriosclerotics, anti-inflammatories, anticoagulants, antihypotensives, antihypoglycemics, antihypertonics, antifibrinolytics, antiepileptics, antiemetics, antidotes, antidiabetics, antiarrhythmics, antianemics, antiallergics, anthelmintics, analgesics, analeptics, aldosterone antagonists and slimming agents.

One possible preparation variant is to dissolve the solubilizer in the aqueous phase, if appropriate with gentle heating and then to dissolve the active ingredient in the aqueous solubilizer solution. The simultaneous dissolution of solubilizer and active ingredient in the aqueous phase is likewise possible.

The use of the copolymers according to the invention as solubilizer can, for example, also be carried out by dispersing the active ingredient in the solubilizer, if appropriate with heating, and mixing it with water with stirring.

In addition, the solubilizers can also be processed in the melt with the active ingredients. In particular, solid solutions can be obtained in this way. Of suitability for this is, inter alia, also the melt extrusion process. Another way of preparing solid solutions is also to prepare solutions of solubilizer and active ingredient in suitable organic solvents and then to remove the solvent by customary methods.

The invention thus also generally provides pharmaceutical preparations which comprise at least one of the copolymers according to the invention as solubilizer. Preference is given to those preparations which, besides the solubilizer, comprise a pharmaceutical active ingredient which is insoluble or sparingly soluble in water, for example from the abovementioned areas of indication.

Of the abovementioned pharmaceutical preparations, particular preference is given to those which are orally applicable formulations.

The content of solubilizer according to the invention in the pharmaceutical preparation is, depending on the active ingredient, in the range from 1 to 75% by weight, preferably 5 to 60% by weight, particularly preferably 5 to 50% by weight.

In the case of solid solutions, the weight ratio of solubilizer to active ingredient may be from 1:1 to 4:1.

### Solubilizers for Food Preparations:

Besides the use in cosmetics and pharmacy, the copolymers according to the invention are also suitable as solubilizers in the food sector for nutrients, auxiliaries or additives which are insoluble or sparingly soluble in water, such as, for example, fat-soluble vitamins or carotenoids. Examples which may be mentioned are clear drinks colored with carotenoids.

### Solubilizers for Crop Protection Preparations:

The use of the copolymers according to the invention as solubilizers in agrochemistry can comprise, inter alia, formulations which comprise pesticides, herbicides, fungicides or insecticides, especially also those preparations of crop protection compositions which are used as spray mixtures or pouring mixtures.

The copolymers according to the invention are notable for a particularly good solubilizing effect. It is also advantageous if they are solids which have relatively favorable properties from a processing point of view.

The examples below illustrate the preparation and use of the copolymers according to the invention in more detail.

### Preparation of the Vinyllactam Prepolymers

VP: N-Vinylpyrrolidone; VAc: Vinyl acetate

### General Procedure

Initial charge: 10 g of feed 1,200 g of methanol

### Feed 1: VP, VAc

Feed 2: tert-butyl perpivalate (75% strength by weight in aliphatics mixture), 67 g of methanol
Feed 3: tert-butyl perpivalate (75% strength by weight in aliphatics mixture), 200 g of methanol
Feed 4: sodium methoxide, 5 g of methanol
For precise quantitative data see the table below.

The preparation was carried out in a stirred apparatus under a nitrogen atmosphere. The initial charge was heated to 65° C., admixed with 3.4 g of feed 2 and polymerized for 15 min. Then, feed 1 and the remainder of feed 2 were started and added over a period of 4 hours. Afterwards, feed 3 was added as a batch and the mixture was after-polymerized for two hours. Then, feed 4 was added at 58° C. and the reaction mixture was kept at this temperature for one hour. Methanol and methyl acetate which formed were distilled off and the product was subjected to steam distillation. After steam distillation, a clear yellowish solution was obtained from which the copolymer was obtained by freeze-drying.

| | | Amounts of feed substances [g] | | | | | |
|---|---|---|---|---|---|---|---|
| Prepolymer | | | | | Initiator | | K value |
| Ex. | VP:VAc | | | | Feed | Feed | 1% strength by |
| No. | [mol %] | VP | VAc | NaOMe | 2 | 3 | weight in water |
| A | 98:2 | 196 | 4 | 0.14 | 1.33 | 1.33 | 49.1 |
| B | 96:4 | 192 | 8 | 0.27 | 1.33 | 1.33 | 52 |
| C | 98:2 | 196 | 4 | 0.14 | 2.66 | 1.33 | 43.2 |
| D | 96:4 | 192 | 8 | 0.27 | 2.66 | 1.33 | 41.8 |

The molecular weight was determined by means of size exclusion chromatography with dimethylacetamide+0.5% by weight LiBr as solvent (temperature: 80° C., flow rate: 1 ml/min, concentration of the solution: 5 g/l). The GPC column was calibrated using PMMA standard (M=800-1,820,000 g/mol).

| | M_{w} [g/mol] | Mₙ [g/moL] | M_{w}/Mₙ |
|---|---|---|---|
| Prepolymer A | 145 000 | 21 000 | 6.9 |
| Prepolymer B | 170 000 | 25 000 | 6.8 |
| Prepolymer C | 87 000 | 14 000 | 6.2 |
| Prepolymer D | 79 000 | 15 000 | 5.3 |

### Preparation of the Copolymers: Examples 1 to 16

### General Procedure:

25 mmol of isophorone diisocyanate, 200 g of solvent and 5 mmol of dibutyltin dilaurate were initially introduced and brought to the desired temperature. Then, 25 mmol, based on OH groups, of the polyalkylene oxide were added and the mixture was maintained at the set temperature until the isocyanate value had dropped to 50%. Then, 25 mmol, based on OH groups, of the vinyllactam prepolymer, were added and the temperature was increased to the chosen reaction temperature.

The hydroxyl numbers of the polyalkylene oxides and vinyllactam prepolymers are determined by acetylation of the hydroxyl groups with acetic anhydride and subsequent titration of the resulting acetic acid with base (DIN 53240 and DIN 16945, see Römpp, 9th edition).

The isocyanate value was determined titrimetrically: 1 g of the product was dissolved in 20 ml of a 0.1 molar solution of dibutylamine in toluene, and back-titrated with 0.1 molar hydrochloric acid using bromophenol blue as indicator.

| | | | Temp. of | |
|---|---|---|---|---|
| | Polyalkylene oxide | | IPDI | Vinyllactam |
| Ex. No. | used | Solvent | conv. [0° C.] | prepolymer |
| 1 | Kerocom 3271 | Acetone/ | 57 | C |
| | | DMSO | | |
| 2 | Pluronic PE 3100 | Acetone/ | 57 | C |
| | | DMSO | | |
| 3 | Pluriol A 2000 E | DMSO | 100 | C |
| 4 | Kerocom 3271 | DMSO | 100 | D |
| 5 | Pluronic PE 3100 | DMSO | 100 | D |
| 6 | Pluriol A 1350 P | DMSO | 20 | C |
| 7 | Pluriol P 2000 | DMSO | 100 | D |
| 8 | Pluriol A 1350 P | DMSO | 20 | D |
| 9 | Pluriol P 2000 | DMF | 100 | C |
| 10 | Pluriol P 2000 | DMF | 100 | D |
| 11 | Pluronic PE 6100 | Acetone | 57 | C |
| 12 | Pluronic PE 6100 | Acetone | 57 | D |
| 13 | Pluronic PE 6200 | Acetone | 57 | C |
| 14 | Pluronic PE 6200 | Acetone | 57 | D |
| 15 | Pluronic PE 10100 | Acetone | 57 | C |
| 16 | Pluronic PE 10100 | Acetone | 57 | D |

| | | |
|---|---|---|
| Polyalkylene | | |
| oxides used | | |
| (AlkO) | | |
| Pluriol | A 1350 P | 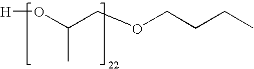 |
| | A 2000 E | 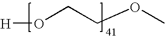 |
| | P 2000 | 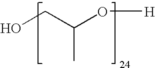 |
| Pluronic | PE 3100 | 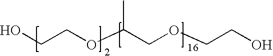 |
| | PE 6100 | 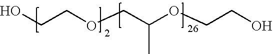 |
| | PE 6200 | 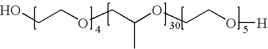 |
| | PE 10100 | 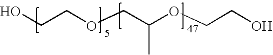 |
| Kerocom | 3271 | 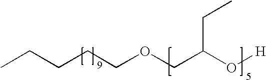 |

| | Gross composition in mol %, based on use | | | | Mol. w. |
|---|---|---|---|---|---|
| Example | amounts | | | | determined by |
| No. | VP | VAc | IPDI | AlkO | GPC |
| 1 | 83.1 | 1.69 | 1.69 | 13.52 | 103 000 |
| 2 | 81.03 | 1.65 | 1.65 | 15.67 | 200 000 |
| 3 | 72.11 | 1.47 | 1.47 | 24.95 | 215 000 |
| 4 | 70.66 | 2.93 | 2.93 | 23.48 | 91 000 |
| 5 | 67.68 | 2.81 | 2.81 | 26.70 | 174 000 |
| 6 | 66.27 | 1.35 | 1.35 | 31.03 | 113 000 |
| 7 | 55.90 | 2.32 | 2.32 | 39.46 | 197 000 |
| 8 | 49.06 | 2.04 | 2.04 | 46.86 | 107 000 |
| 9 | 72.12 | 1.47 | 1.47 | 24.94 | 175 000 |
| 10 | 55.88 | 2.32 | 2.32 | 39.48 | 228 000 |
| 11 | 74.87 | 1.52 | 1.52 | 23.09 | 171 000 |
| 12 | 59.33 | 2.46 | 2.46 | 35.75 | 219 000 |
| 13 | 69.57 | 1.42 | 1.42 | 27.59 | 177 000 |
| 14 | 52.82 | 2.19 | 2.19 | 42.80 | 233 000 |
| 15 | 63.3 | 1.29 | 1.29 | 34.02 | 188 000 |
| 16 | 45.79 | 1.9 | 1.9 | 50.41 | 258 000 |

### Preparation of Solubilizates

2 g of the copolymer were weighed into a beaker. Then, one medicament in each case was weighed into the mixture as follows in order to obtain a supersaturated solution. (If the weighed-in mass dissolved in the medium, the initial weight was increased until a sediment formed).

Amount of active ingredient added: 17-β-estradiol 0.2 g; piroxicam 0.2; clotrimazol 0.2 g; carbamazepine 0.3 g; ketoconazole 0.25 g; griseofulvin 0.25 g; cinnarizine 0.25 g.

Phosphate buffer pH 7.0 was then added until solubilizer and phosphate buffer were present in the weight ratio 1:10. Using a magnetic stirrer, this mixture was stirred at 20° C. for 72 hours. There then followed a resting time of at least one hour. Following filtration of the mixture, it was measured photometrically and the content of active ingredient was determined.

| | Copolymer as in Ex. No./Solubilization at 20° C. in [g/100 ml] | | | | | | | |
|---|---|---|---|---|---|---|---|---|
| Carbamazepine | 1/0.08 | 2/0.07 | 3/0.07 | 4/0.08 | 5/0.08 | 6/0.09 | 7/0.11 | 8/0.20 |

| | Copolymer as in Ex. No./Solubilization at 20° C. in | | | |
|---|---|---|---|---|
| | [g/100 ml] | | | |
| Estradiol | 1/0.01 | 3/0.05 | 5/0.02 | 6/0.16 |

| | Copolymer as in Ex. No./Solubilization at 20° C. in [g/100 ml] | | | | | | | |
|---|---|---|---|---|---|---|---|---|
| Piroxicam | 1/0.29 | 2/0.48 | 3/0.47 | 4/0.32 | 5/0.29 | 6/0.33 | 7/0.39 | 8/0.36 |

1. A vinyllactam copolymer modified with polyalkylene oxide side-chains.

2. The copolymer according to claim 1, wherein the copolymer is produced by free-radically initiated copolymerization of N-vinyllactams with monoolefinically unsaturated comonomers which carry hydroxyl or amino groups or comprise a group which can be converted into hydroxyl or amino groups by hydrolysis, and subsequent coupling of the polyalkylene oxide side-chains onto the amino or hydroxy groups of the vinyllactam copolymer.

3. The copolymer according to claim 1, wherein the monoolefinically unsaturated comonomers monomers chosen from the group consisting of vinyl esters of C₂-C₈-carboxylic acids, monovinyl ethers of C₂-C₈-diols, vinylamides, hydroxyalkyl and aminoalkyl esters of acrylic acid and of methacrylic acid.

4. The copolymer according to claim 1, wherein the vinyllactam copolymers are obtained by free-radically initiated copolymerization of N-vinyllactams and vinyl acetate with subsequent saponification of the ester groups.

5. The copolymer according to claim 1, wherein the N-vinyllactam used is N-vinylpyrrolidone.

6. The copolymer according to claim 1, wherein the vinyllactam copolymer is obtained by reacting 50 to 99.9 mol % of N-vinyllactams and 0.1 to 50 mol % of comonomers.

7. The copolymer according to claim 1, wherein the vinyllactam copolymer is obtained by reacting 80 to 99 mol % of N-vinyllactams and 1 to 20 mol % of comonomers.

8. The copolymer according to claim 1, wherein the polyalkylene oxides have molecular weights of from 500 to 20,000 daltons.

9. The copolymer according to claim 1, wherein the polyalkylene oxides are chosen from the group consisting of polyethylene glycols, polypropylene glycols, polybutylene glycols and monoalkoxylated derivatives thereof.

10. The copolymer according to claim 1, wherein the polyalkylene oxides used are polyoxyethylene-polyoxypropylene block copolymers.

11. The copolymer according to claim 1, wherein the coupling of the polyalkylene oxide side-chains takes place via diisocyanates.

12. The copolymer according to claim 1, wherein the polyalkylene oxides are functionalized by reaction with a diisocyanate before reaction with the vinyllactam copolymer.

13. The copolymer according to claim 1, wherein the polyalkylene oxides are used in equimolar amounts, based on the amino or hydroxyl groups of the vinyllactam copolymer.

14. The copolymer according to claim 1, wherein the diisocyanate used is isophorone diisocyanate.

15. The copolymer according to claim 1, wherein the coupling of the polyalkylene oxide side-chains takes place by direct alkoxylation with alkylene oxides.

16. The copolymer according to claim 1, wherein the copolymers have molecular weights M_{w} of from 20,000 to 250,000 g/mol.

17-24. (canceled)

25. A preparation of substances which are sparingly soluble in water, comprising, as solubilizers, copolymers according to claim 1.

26. The preparation according to claim 25, comprising a biologically active substance as substance which is sparingly soluble in water.

27. The preparation according to claim 25, comprising a pharmaceutical active ingredient as biologically active substance which is sparingly soluble in water.

28. The preparation according to claim 27 in the form of orally applicable administration forms.

29. The preparation according to claim 25, comprising a cosmetic active ingredient as biologically active substance which is sparingly soluble in water.

30. The preparation according to claim 25, comprising an agrochemical active ingredient as biologically active substance which is sparingly soluble in water.

31. The preparation according to claim 25, comprising a food supplement or a dietetic active ingredient as biologically active substance which is sparingly soluble in water.

32. The preparation according to claim 25, comprising a dye as substance which is sparingly soluble in water.
